(19) United States
(12) Patent Application Publication
(10) Pub. No.: US 2005/0090512 A1
(43) Pub. Date: Apr. 28, 2005

(54) Method of treating extreme physical or mental stress using L-theanine to obtain accelerated regeneration

(76) Inventors: Kurt-Reiner Geiss, Langen (DE); Michael Weiss, Paderborn (DE); Nagahiro Yamazaki, Yokkaichi (JP); Lekh Raj Juneja, Yokkaichi (JP); Makoto Ozeki, Yokkaichi (JP)

Correspondence Address: FLEIT KAIN GIBBONS GUTMAN & BONGINI, COURVOISIER CENTRE II, SUITE 404, 601 BRICKELL KEY DRIVE, MIAMI, FL, 33131, US

(21) Appl. No.: 10/695,427

(22) Filed: Oct. 28, 2003

(51) Int. Cl. A61K 31/522

(52) U.S. Cl. 514/263.37

(57) ABSTRACT

Method of using L-Theanine for acceleration of regeneration after stressing. A quantity of at least 50 mg of L-Theanine is administered after physical or mental stressing. For example, L-Theanine can be administered in the form of a food stuff, such as a functional food with L-Theanine additive, or in the form of a complete drink.

### BACKGROUND OF THE INVENTION

1. Field of the Invention

The present invention relates to the field of usage of L-Theanine, and more particularly without limitation, to usage of L-Theanine for functional foods.

2. Prior Art

The indications of L-theanine for the treatment of obesity, symptoms of anxiety, premenstrual syndrome, sensitivity to cold, menopausal disturbances, sleep disturbances, dyspnoea and further medical indications are known from European application 1 057 483. For treatment, L-theanine is administered in a dose of 0.2 to 2.0 mg per kilogram per day, in particular 0.2 to 300 mg per kilogram of body weight per day, and specifically in a combination with minerals, such as for example iron, magnesium, copper, zinc, selenium, calcium or other minerals.

A further medical indication of L-theanine for the treatment of mental and physical diseases as a result of stress is known from Japanese application 06 100 442. For the treatment, L-theanine may be administered in the form of a drink.

It is known from Japanese application 09 012 454 that a dose of 0.3 to 300 mg of L-theanine per kilogram of body weight, preferably 0.3 to 300 mg per kilogram of body weight, has an effect on the production of alpha-waves, so that learning ability is increased.

A process for producing L-theanine is also known from Japanese application 2000 026 383.

A process for increasing the functional capacity of the brain of a person and a device for carrying out this process, which is not based on the administration of a pharmacological active ingredient, but on the physical stress of the person with the aid of an ergometer, is known from German Patentschrift 4 102 031. For the treatment of the person, a physical stress in the range between 50% and 65% of the physical stress at the threshold of the aerobic to anaerobic region is induced.

It is known that one administration of 50 to 200 mg of L-theanine leads to stimulation of the production of alpha-waves from “L-Theanine—a unique amino acid of green tea and its relaxation effects in humans”, Trends in Food Science and Technology, Volume 10, No. 6/7, 1999. This knowledge has been used in the commercially available product “Sun-Theanin”, which may be used as a relaxant and as an agent for the reduction of stress.

### SUMMARY OF THE INVENTION

The present invention provides a method for treating a person under extreme stress, either physical or mental, with at least 50 mg of L-Theanine for purpose of acceleration of regeneration. The invention in effect constitutes a new use of ingesting L-Theanine after physical and/or mental stressing to accelerate regeneration. In accordance with the present invention a quantity of at least 50 mg of L-Theanine is administered after stressing. Preferably a quantity of not more than 200 mg of L-Theanine is administered after stressing.

In accordance with a preferred embodiment of the invention, L-Theanine is administered to a person in the form of a food stuff with L-Theanine as an additive. For example, the food stuff is a functional food having an L-Theanine content between 50 mg and 200 mg per portion.

In accordance with a further preferred embodiment of the invention, L-Theanine is administered in the form of a complete drink having an L-Theanine content of about 100 mg, or about 600 mg per litre.

The invention concerns a method of treatment comprising the step of introducing into a person experiencing physical and/or mental stressing a quantity of at least 50 mg of L-theanine for accelerating the person's regeneration from the stressing. The L-theanine can be administered to the person. The quantity of L-theanine is not greater than 200 mg.

The method of the invention can be carried out by administering L-theanine in the form of a foodstuff with L-theanine as an additive. Also, the method of the invention can be carried out using a foodstuff that is a functional food notionally divisible into a plurality of preselected portions, with each said preselected portion having an L-theanine content of from about 50 mg to about 200 mg.

The method of the invention can be carried out using L-theanine administered in the form of a complete drink having an L-theanine content of about 100 mg or of about 600 mg per liter.

In the method of the invention, the L-theanine is selected from the group consisting of an enzymatically recovered r-ethylamino-L-glutamine, natural L-theanine and mixtures thereof.

In accordance with a further preferred embodiment of the invention the L-Theanine is an enzymatically recovered y-ethylamino-L-glutamine or natural L-theanine.

It is to be noted that the present invention provides a method to substantially accelerate the natural regeneration process after severe or extreme physical and/or mental stressing of a human.

Usually the duration which is required for full regeneration after stressing is about one to two hours. In accordance with the invention, a dose of at least 50 mg L-Theanine is administered after the stressing by the person ingesting or drinking a food containing the L-Theanine. This way the regeneration process is substantially accelerated. For example the natural regeneration process can be shortened to about 30 minutes.

### BRIEF DESCRIPTION OF THE DRAWING

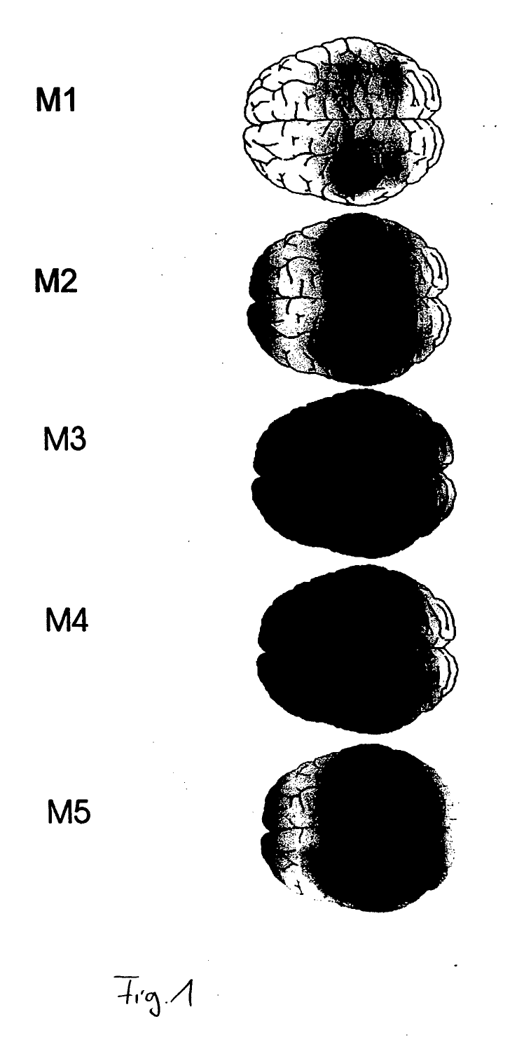

In the following, preferred embodiments of the invention will be described in greater detail by way of example with reference to the sole drawing, FIG. 1, which is illustrative of the acceleration of the regeneration of a human after stressing.

### DETAILED DESCRIPTION PREFERRED EMBODIMENTS OF THE INVENTION

In animal tests, in addition to other effects of L-theanine, suppression of the effects of caffeine on the central nervous system has also been observed. In the search for the mechanisms responsible for this, various investigators came upon influences of the neurotransmitter systems serotonin, dopamine and noradrenalin, which are responsible, inter alia, for alertness, motivation, drive and vigilance and when they are not functioning correctly, motor and psychological disturbances, such as depression and Parkinson's disease may appear. Now however, animal tests can hardly be transferred to the human being, the doses used were always extremely high and the isolated investigation of individual neurotransmitter systems hardly indicates the reactions to be expected in vitro due to the prevailing interactions. Pilot observations pointed to relaxing, relieving effects particularly in characteristically anxious test people, which, indicated by alpha-wave activity in an EEG, was also objectified by physiological measuring processes.

In order to verify this and possible further effects of L-theanine on different body functions in the human being in quantities which are conventional hitherto for dose, an investigation model having corresponding physical and biochemical detection processes was consequently needed, which has been developed on the basis of considerations regarding the physiological connections.
(a) the procedure is based on the hypothesis that the substance to be tested brings influence to bear on stress management and copying,
(b) improves relaxation ability and recovery (recreation) after a stress situa tion,
(c) supports the natural switching mechanisms from strain to relaxation (from ergotropy to trophotropism).

Based on the knowledge derived from investigations on animals and the hypothesis of a relaxing (stress-relieving) effect, the following are to be recorded on body functions:
central nervous activity
peripheral adaptation reactions by stress hormones
indications regarding the coupling between central nervous control and hormonal regulation
circulatory behaviour
electrodermal stress reaction, optionally wellbeing

Electrophysiological processes and measurement of parameters in the blood and in the urine are suitable to record the functions in these regions. The following detection processes are used in particular:
recording of brain current graphs (electroencephalography/EEG) with mathematic processing according to frequency, performance and localisation (topographical spectral performance by means of EEG mapping);
blood level of stress hormones and possibly deposition quantity of hormone degradation products in the urine;
production of correlations between central nervous parameters and hormone levels in the blood;
heart-rate recording and blood-pressure measurement.

Skin Resistance Measurements (Electrosympathicography), Optionally use of Evaluated Questionnaires for Measurement of Wellbeing

The investigation model is based on the production of physical stress by means of almost maximum bicycle ergometry as a method which is independent of surroundings, reproducible, reliable and exactly meterable individually according to a pre-test, and which triggers well-researched adaptation reactions. After stress, a natural relaxation process starts (down-regulation), which forms the basis for comparison in the placebo test and thus may be used for differentiation between support of natural mechanisms by the test substance and possible non-physiological but pharmacological effects of the test substance. The target parameters in the after-stress phase are observed after administration of the test substance (optionally in different dose)/of a placebo in randomised sequence in a double-blind process.

Measurement of the hypohysen hormone prolactin in the blood serum, which after physical stress reacts like a stress hormone, plays a particular part in the selection of the hormonal parameters, since it is under the control of the central neurotransmitters dopamine (inhibits secretion) and serotonin (promotes release) and thus may reflect the central situation of these two systems. The concentrations of the catecholamines dopamine, noradrenalin and adrenalin in the blood plasma with their different origins (sympathetic ganglia, adrenal medulla) and their effects on circulation and metabolism are selected as further stress parameters from the peripheral ergotropic sympathetic system, and the serum level of the metabolism-stabilising and immune function-controlling adrenal cortex hormone cortisol. Measurement of serotonin in the blood serum may be used by way of supplement, even if the origin of the serotonin measured in the periphery cannot be assigned exactly. For longer observation periods, the rates of deposition of the degradation products of catecholamines and serotonin in the urine may also be informative.

Investigation Design and Methods

The bicycle ergometry is effected as a multi-stage test with increase up to near maximum functional capacity. The stage height and the maximum performance is determined in a pre-test, which proceeds starting from 50 watts with increase by 50 watts every 3 minutes up to physical exhaustion. The last stage lasting 3 minutes is the criterion for the actual test stress. It is reached there in 4 equal incremental stages each of 3 minutes and then as the fifth stage, 4 minutes are taken. (Variation possibilities: ramp stress, maximum steady state).

The first measurement is effected immediately after the end of stress, the test drink is then administered and the recovery phase introduced in standardised manner (usually while lying in a separate peaceful darkened room). Further measurements are effected up to 2 hours after administration of the drink, wherein focus must be directed towards the known or to be foreseen uptake and distribution rate of the test substance into the brain.

Results of the Investigation on the Effect of L-Theanine-Containing Drinks

Drinks with 0 and 50 and 200 mg of L-theanine tasting the same and looking identical were investigated in double-blind manner controlled by placebo under exactly the same conditions according to the above investigation model.

Due to physical near maximum stress, there is in the EEG a rise in electrical performance in all frequency ranges and displacement in the spectral performance density to higher frequencies. The typical fall in spectral electrical performance averaged over all electrode positions in the international 10:20 system was not changed by the L-theanine-containing drinks. The natural switching mechanism from work to recovery with a down-regulation in the rapid frequency ranges was thus not influenced globally. Even if no significant differences were calculable mathematically at the individual measuring points in time, the qualitative consideration of the graphs and the glow-mode representation of the maps did give indications of a temporal acceleration of the processes. When calculating the electrical performance at the individual derivation points, related percentage-wise to the value directly after the end of stress, lower values could however be found in the alpha-2 wave range with a mathematical probability of 90% directly in front of and behind the central groove (in the vicinity of the motor and sensory central convolution) 30 minutes after the drink (44 minutes after the end of stress). At the same point in time, highly significant lower values could be detected in the beta-1 range above the central parietal brain (electrode Pz) and in the beta-2 range above the left occiput (electrode O1). In the further course, there were then no longer any differences in the rapid alpha and beta wave ranges speaking for activation and stress, which means that the down-regulated values were reached sooner using L-theanine-containing drinks.

In the case of the serum hormone values of catecholamines, serotonin and cortisol and also in the case of the depositions of the degradation products of serotonin and catecholamines in the urine, no differences between the drinks could be determined, however multi-factorial variance analysis showed significant differences after 200 mg of L-theanine in the overall course of the prolactin values compared to 0 and 50 mg of L-theanine, with 50 mg of L-theanine being the lower limit of efficacy. The results of the skin resistance measurements were not influenced by the drink factor.

In the case of the correlations between frequency-related averaged brain current activities, the correlations with dopamine disappeared in the tests with L-theanine in the slow wave range and instead there were correlations of adrenalin with activities in the rapid wave range as well as new negative correlations of alpha-1 waves with cortisol and prolactin. The correlations of beta-1 performance with prolactin and cortisol disappeared at the individual electrode positions, in which in the temporal sequence, significant differences of the electrical performances related percentage-wise to the starting values had appeared, namely Pz and O1 (see above).

### Conclusions

Regarding the Process

Typical sequences after reproducible and individually controllable physical stress are rendered visible by the process. The influence of the natural switching process from activity to recovery (from ergotropy to trophotropism) by foodstuffs or medicines can be understood both in the sense of support of the physiological mechanisms, as shown by the example of L-theanine, or also optionally in the sense of pharmacological influence with changes to the fundamental courses of the measured values of electrical brain activity and hormonal control of body functions.

Regarding L-Theanine

L-Theanine in the dose range 50 to 200 mg does not trigger quantitative and fundamental changes in physiological sequences of down-regulation after stress in the pharmacological sense, but acts to accelerate the processes of switching from stress to recovery, thus supports switching into the relaxation phase after stress in the sense of promoting regeneration. Due to the changed correlations and the effect on the prolactin level, analogously to results from animal tests known from the literature, it may be assumed that the mechanisms lie in the central neurotransmitter system and at the switch points between central electrical brain activity and the peripheral hormonal control and regulating system. The accelerated drop in activity in the rapid electrical frequency ranges in the areas of processing sensory stimuli and in the region of switching electrical performances in the cerebral cortex to hormonal regulation proposals, the shift to other stress hormones but while retaining the hormonal reactivity, is presumably particularly relaxation-promoting.

According to the teaching of the invention, L-theanine may thus on the one hand be added to a foodstuff, such as for example a drink, yoghurt or dietetic foodstuffs, in a concentration of at least 50 mg per administration or portion, so that after consumption of the foodstuff, a regenerative and/or relaxing and/or calming effect starts. For example, L-theanine may be added to a complete drink in a quantity of 100 mg or 600 mg per litre.

On the other hand, L-theanine may also be used in a dose of over 200 mg per administration as medicament for the treatment of nervousness and internal anxiety. The use of L-theanine in a dose of over 200 mg thus permits the production of a medicament for the treatment of nervousness and/or internal anxiety, wherein each conventional form of administration may be selected—such as for example as a capsule, coated tablet or effervescent tablet.

In principle both natural and synthetically recovered L-theanine is suitable both for the said purposes relating to nutritional physiology and pharmacological purposes. L-theanine is an amino acid of natural origin, which may be recovered, for example from green tea. On the other hand, L-theanine may also be recovered enzymatically as γ-ethylamino-L-glutamine.

FIG. 1 shows a human brain at different times M1, M2, M3, M4 and M5 after physical or mental stressing. FIG. 1 illustrates the distribution of delta, alpha2 and beta1 waves in the brain after stressing at the various stages during the regeneration process, i.e. at times M1 to M5. Time M1 is immediately after stressing; time M5 is showing the brain at the end of the regeneration process. The time M3 is in a state of drowsiness. At times M2 and M4 the brain is in a transitional state, i.e. at time M2 it transitions from the state of exhaustion (time M1) to the state of drowsiness (time M3) and at time M4 it transitions from the state of drowsiness (time M3) to complete regeneration (time M5).

Usually this regeneration process of the human brain after stressing takes between one and two hours. When a dose of at least 50 mg L-Theanine is administered after the stressing, the natural regeneration process is substantially accelerated and takes only about 30 minutes. This substantial acceleration of the natural regeneration process after stressing is due to the acceleration of the physiological regeneration mechanisms by the L-Theanine.

1. Method of treatment comprising the step of introducing into a person experiencing physical and/or mental stressing a quantity of at least 50 mg of L-theanine for accelerating the person's regeneration from the stressing.

2. Method of claim 1 wherein the L-theanine is administered to the person.

3. Method of claim 2, wherein the quantity of L-theanine is not greater than 200 mg.

4. Method of claim 2, wherein L-theanine is administered in the form of a foodstuff with L-theanine as an additive.

5. Method of claim 4, wherein the foodstuff is a functional food notionally divisible into a plurality of preselected portion, with each said preselected portion having an L-theanine content of from about 50 mg to about 200 mg.

6. Method of claim 2, wherein L-theanine is administered in the form of a complete drink having an L-theanine content of about 100 mg or of about 600 mg per liter.

7. Method of claim 1, wherein the L-theanine is selected from the group consisting of an enzymatically recovered y-ethylamino-L-glutamine, natural L-theanine and mixtures thereof.
